(19) United States
(12) Patent Application Publication
(10) Pub. No.: US 2009/0177011 A1
(43) Pub. Date: Jul. 9, 2009

(54) PROCESS FOR THE PRODUCTION OF ANILINES

(75) Inventors: Harald Walter, Basel (CH); Camilla Corsi, Basel (CH); Josef Ehrenfreund, Basel (CH); Clemens Lamberth, Basel (CH); Hans Tobler, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: Syngenta Crop Protection, Inc., Greensboro, NC (US)

(21) Appl. No.: 11/720,692

(22) PCT Filed: Dec. 8, 2005
(86) PCT No.: PCT/EP05/13167

§ 371 Date: Jun. 1, 2007

(30) Foreign Application Priority Data
Dec. 10, 2004 (CH) 02050/04

(51) Int. Cl. C07C 209/06

(52) U.S. Cl. 564/407

(57) ABSTRACT

The present invention relates to a process for the preparation of compounds of formula (I) wherein R₁, R₂ and R₃ are each independently of the others hydrogen or methyl, by reaction of compounds of formula (II) wherein R₁, R₂ and R₃ are as defined for formula (I) and X is bromine or chlorine, with ammonia in the presence of a catalytic amount of at least one copper-containing compound.

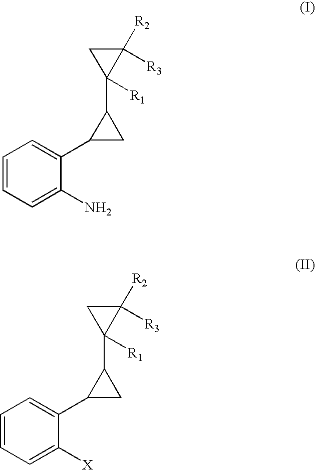

The present invention relates to a process for the amination of ortho-alkyl-substituted halobenzenes and to the use of ammonia and copper-containing compounds in the amination of ortho-alkyl-substituted halobenzenes.

Ortho-alkyl-substituted primary anilines, for example 2-bicyclopropyl-2-yl-phenylamine, are valuable intermediates in the preparation of fungicides, as described, for example, in WO 03/074491.

The preparation of primary arylamines from the corresponding aryl halides using ammonia in the presence of copper-containing catalysts has been known for a long time and is described, for example, in Berichte der deutschen Chemischen Gesellschaft, 69, 1534-1537 (1936), in Journal of Organic Chemistry, 64, 6724-6729 (1999) and in Tetrahedron Letters, 42, 3251-3254 (2001). One possible amination mechanism proceeds by way of a nucleophilic attack on the aromatic nucleus of the aryl halide (possible mechanisms are discussed in: Tetrahedron, 40, 1433-1456 (1984)). It is generally known that such reactions proceed in high yields only with electron-poor heteroaryl nuclei, for example the pyridine nucleus, or with unsubstituted benzene nuclei or with activated benzene nuclei of reduced electron density. An example of such a benzene nucleus of reduced electron density is a nucleus having a nitro group in the ortho- or para-position to the halogen atom being displaced.

Performing such copper-catalysed amination with deactivated benzene nuclei, such as, for example, ortho-alkyl-substituted halobenzenes, with a high yield is regarded in the specialist literature as being extremely difficult. For example, the standard works of specialist literature propose exclusively unsubstituted or activated aryl halides as starting materials for copper-catalysed amination (see, for example, Tetrahedron, 40 (1984), page 1433 and pages 1435-1436 and Chemical Reviews, 49 (1951) pages 392 and 395).

Only the Journal of Organic Chemistry, 64, 6724-6729 (1999) describes the use of a copper/copper(I) chloride catalyst for the amination of a halobenzene which is substituted in the ortho-position by a 1,2,3,4-tetrahydro-isoquinoline derivative. In that process, however, copper powder is used, which is very expensive; a long reaction period of 5 days is required and large amounts of catalyst are needed. For those reasons, such a process is particularly unsuitable for large-scale preparation of ortho-alkyl-substituted primary anilines.

Modern processes for the preparation of ortho-alkyl-substituted anilines therefore utilise palladium-containing catalysts. The successful use of palladium-containing catalysts in the amination of deactivated halobenzenes is known and is described, for example, for a number of ortho-alkyl-substituted bromobenzenes or chlorobenzenes, such as, for example, 2-bromotoluene, in Journal of Organic Chemistry, 64, 5575-5580 (1999) and in Journal of Organic Chemistry, 65, 1158-1174 (2000).

The disadvantage of palladium-catalysed amination technology is that direct preparation of primary anilines is not possible. For the preparation of primary anilines a further reaction step is necessary. Such a two-step process for the preparation of primary anilines is described in WO 03/074491. According to WO 03/074491, ortho-alkyl-substituted primary anilines can be prepared by reacting the corresponding ortho-alkyl-substituted halobenzenes in a two-step reaction first with benzophenone-imine in a palladium(II)-catalysed reaction and then reacting the reaction products with hydroxylamine hydrochloride and sodium acetate or with acids, for example hydrochloric acid.

Such a reaction procedure for the preparation of primary anilines is particularly unsuitable for the large-scale preparation of ortho-alkyl-substituted primary anilines, however, on account of the need for a second process step and on account of the expensive palladium-containing catalysts.

The aim of the present invention is therefore to provide a novel process for the preparation of ortho-alkyl-substituted primary anilines that avoids the above-mentioned disadvantages of the known processes and makes it possible to prepare those compounds in high yields and good quality in an economically advantageous and easily handled way.

The present invention accordingly relates to a process for the preparation of compounds of formula I

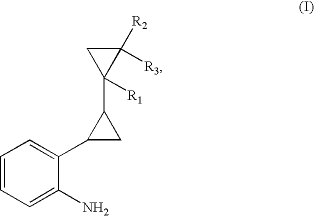

wherein R₁, R₂ and R₃ are each independently of the others hydrogen or methyl, by reaction of a compound of formula II

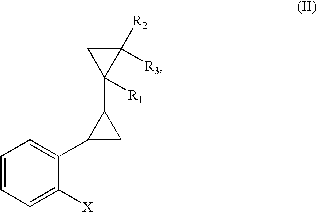

wherein R₁, R₂ and R₃ are as defined for formula I and X is bromine or chorine, with ammonia in the presence of a catalytic amount of at least one copper-containing compound.

Compounds of formula I occur in various stereoisomeric forms, which are depicted in formulae I_{I}, I_{II}, I_{III} and I_{IV}

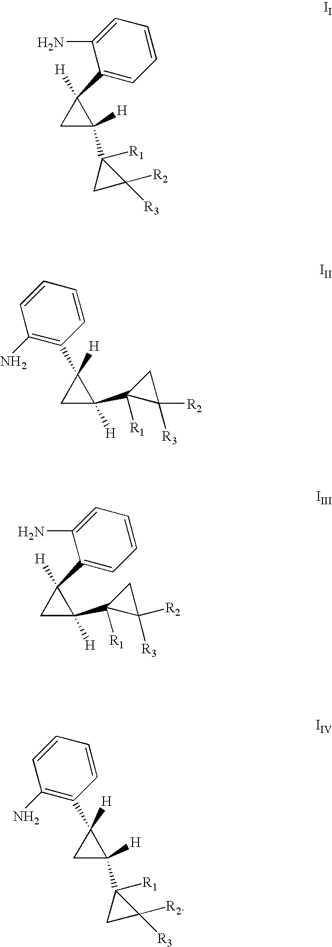

The process according to the invention includes the preparation of those stereoisomeric forms of formulae I_{I}, I_{II}, I_{III} and I_{IV} wherein R₁, R₂ and R₃ are as defined for formula I, and the preparation of mixtures of those stereoisomeric forms in any ratio.

Compounds of formula Ia (trans)

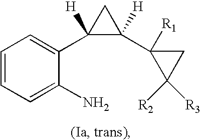

wherein R₁, R₂ and R₃ are as defined for formula I, are to be understood in the context of the present invention as being compounds of formula I_{I} wherein R₁, R₂ and R₃ are as defined for formula I; compounds of formula I_{II} wherein R₁, R₂ and R₃ are as defined for formula I; or a mixture in any ratio of compounds of formula I_{I} wherein R₁, R₂ and R₃ are as defined for formula I and compounds of formula I_{II} wherein R₁, R₂ and R₃ are as defined for formula I.

Compounds of formula Ib (cis)

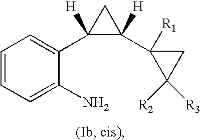

wherein R₁, R₂ and R₃ are as defined for formula I, are to be understood in the context of the present invention as being compounds of formula I_{III} wherein R₁, R₂ and R₃ are as defined for formula I; compounds of formula I_{IV} wherein R₁, R₂ and R₃ are as defined for formula I; or a mixture in any ratio of compounds of formula I_{III} wherein R₁, R₂ and R₃ are as defined for formula I and compounds of formula I_{IV} wherein R₁, R₂ and R₃ are as defined for formula I.

Compounds of formula II occur in various stereoisomeric forms, which are depicted in formulae II_{I}, II_{II}, II_{III} and II_{IV}:

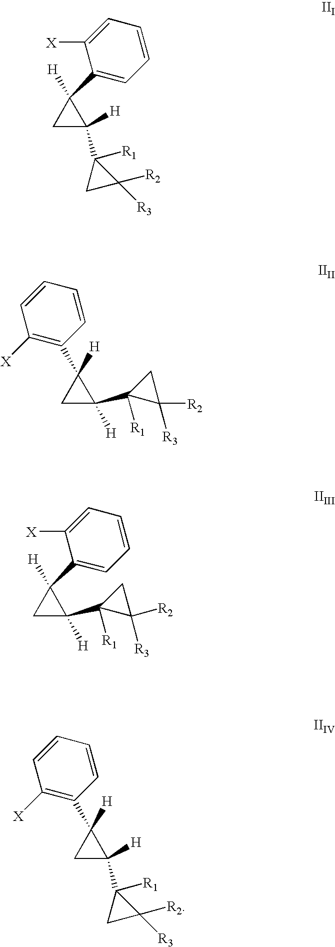

The process according to the invention includes the use of those stereoisomeric forms of formulae II_{I}, II_{II}, II_{III} and II_{IV}, wherein X, R₁, R₂ and R₃ are as defined for formula II, and the use of mixtures of those stereoisomeric forms in any ratio.

Compounds of formula IIa (trans)

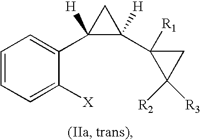

wherein X, R₁, R₂ and R₃ are as defined for formula I, are to be understood in the context of the present invention as being compounds of formula II_{I} wherein X, R₁, R₂ and R₃ are as defined for formula II; compounds of formula II_{II} wherein X, R₁, R₂ and R₃ are as defined for formula II; or a mixture in any ratio of compounds of formula II_{I} wherein X, R₁, R₂ and R₃ are as defined for formula II and compounds of formula II_{II} wherein X, R₁, R₂ and R₃ are as defined for formula II.

Compounds of formula IIb (cis)

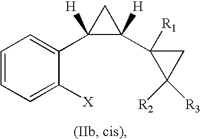

wherein X, R₁, R₂ and R₃ are as defined for formula II, are to be understood in the context of the present invention as being compounds of formula II_{III} wherein X, R₁, R₂ and R₃ are as defined for formula II; compounds of formula II_{IV} wherein X, R₁, R₂ and R₃ are as defined for formula II; or a mixture in any ratio of compounds of formula II_{III} wherein X, R₁, R₂ and R₃ are as defined for formula II and compounds of formula II_{IV} wherein X, R₁, R₂ and R₃ are as defined for formula II.

The process according to the invention is especially suitable for the preparation of compounds of formula I wherein R₁ is hydrogen or methyl; and R₂ and R₃ are hydrogen.

The process according to the invention is more especially suitable for the preparation of compounds of formula I wherein R₁, R₂ and R₃ are hydrogen.

In the process according to the invention it is preferred to use compounds of formula II wherein X is bromine.

Copper-containing compounds include, for example, copper(I) compounds, copper(II) compounds, mixtures of copper(I) compounds, mixtures of copper(II) compounds, mixtures of copper(I) compounds with copper(II) compounds, mixtures of elemental copper with copper(I) compounds and mixtures of elemental copper with copper(II) compounds.

Copper(I) compounds include, for example, copper(I) salts, the use of which is preferred. Suitable copper(I) salts are, for example, CuCl, CuBr, CuI, Cu₂S, copper(I) acetate and Cu₂O, preferably Cu₂O.

Copper(II) compounds include, for example, copper(II) salts, the use of which is preferred. Suitable copper(II) salts are, for example, Cu₂SO₄, Cu₂SO₄×4-6 mol H₂O, CuO, CuS, CuCl₂, CuCl₂×2 mol H₂O and copper(II) acetate.

As a mixture of copper(I) compounds there may be used, for example, a mixture of CuCl and Cu₂O.

In the process according to the invention it is preferred to use copper(I) compounds or mixtures of copper(I) compounds as copper-containing compounds.

In the process according to the invention it is especially preferred to use copper(I) compounds as copper-containing compounds.

In the process according to the invention, copper-containing compounds are used in catalytic amounts. Copper-containing compounds are used preferably in a ratio of from 1:5 to 1:100 relative to compounds of formula II, especially in a ratio of from 1:10 to 1:20.

The reaction according to the invention is carried out at elevated temperature, preferably in a temperature range of from 100° C. to 200° C., especially in a temperature range of from 130° C. to 170° C.

The reaction according to the invention is carried out at elevated pressure, preferably at a pressure of from 20 bar to 150 bar, especially at a pressure of from 35 bar to 85 bar.

The reaction period for the reaction according to the invention is generally from 1 to 48 hours, preferably from 6 to 24 hours, especially from 6 to 18 hours.

The reaction according to the invention can be carried out in an inert solvent; the inert solvent is preferably non-aqueous.

Suitable solvents are, for example, methanol, ethanol, propanol, isopropanol, n-butanol, tert-butanol, ethylene glycol and diethylene glycol. The preferred solvent is ethylene glycol.

In a different preferred embodiment, the reaction according to the invention is carried out without a solvent.

In the reactions according to the invention, ammonia is used in equimolar amounts or in excess relative to compounds of formula II, preferably in an up to 500-fold excess, especially in an up to 200-fold excess, more especially in an 80-fold to 120-fold excess.

In the process according to the invention, ammonia can be introduced into the reaction vessel in liquid form or in gaseous form.

The process according to the invention is very especially suitable for the preparation of compounds of formula I wherein R₁, R₂ and R₃ are each independently of the others hydrogen or methyl, by reaction of a compound of formula II wherein R₁, R₂ and R₃ are each independently of the others hydrogen or methyl and X is bromine, with ammonia in the presence of a catalytic amount of Cu₂O, in a temperature range of from 130° C. to 170° C., with ethylene glycol as solvent, ammonia being used in an 80-fold to 120-fold excess relative to the compound of formula II.

Especially suitable for this embodiment are compounds of formula I wherein R₁ is hydrogen or methyl; and R₂ and R₃ are hydrogen.

Very especially suitable for this embodiment are compounds of formula I wherein R₁, R₂ and R₃ are hydrogen.

The compounds of formula II wherein X is bromine are generally known and can be prepared in accordance with the methods described in WO 03/074491. The compounds of formula II wherein X is chlorine can be prepared analogously in accordance with the methods described in WO 03/074491 for the corresponding compounds of formula II wherein X is bromine.

The present invention relates also to the use of ammonia in the presence of a catalytic amount of at least one copper-containing compound in the amination of compounds of formula II.

The present invention relates also to a process for the amination of compounds of formula II by using ammonia as aminating agent and a catalytic amount of at least one copper-containing compound.

The present invention is illustrated in greater detail with the aid of the following Examples:

### EXAMPLE P1

### Preparation of 2-bicyclopropyl-2-yl-phenylamine

A mixture of 3 g of 2-(2-bromophenyl)-bicyclopropyl (12.7 mmol, trans/cis mixture), 20 g of ammonia gas (1.17 mol), 181 mg of Cu₂O (1.26 mmol) and 20 ml of ethylene glycol is heated at a temperature of 150° C. for 24 hours in an autoclave at a pressure of 34 bar. After evaporation of the ammonia, 200 ml of ethyl acetate are added. The organic phase is washed with water and dried over sodium sulfate and concentrated by evaporation. For separation of secondary products, chromatography is carried out on silica gel (eluant: ethyl acetate/hexane 1:4). After removal of the eluant, 1.47 g of 2-bicyclopropyl-2-yl-phenylamine (67% of theory) are obtained in the form of a brownish liquid (trans/cis ratio: 7:3).

### EXAMPLE P2

### Preparation of 2-(1′-methyl-bicyclopropyl-2-yl)-phenylamine

A mixture of 3 g of 2′-(2-bromophenyl)-1-methyl-bicyclopropyl (11.9 mmol, trans/cis mixture), 20 g of ammonia gas (1.17 mol), 171 mg of Cu₂O (1.19 mmol) and 20 ml of ethylene glycol is heated at a temperature of 150° C. for 24 hours in an autoclave at a pressure of 40 bar. After evaporation of the ammonia, 200 ml of ethyl acetate are added. The organic phase is washed with water and dried over sodium sulfate and concentrated by evaporation. For separation of secondary products, chromatography is carried out on silica gel (eluant: ethyl acetate/hexane 1:4). After removal of the eluant, 1.20 g of 2-(1′-methyl-bicyclopropyl-2-yl)-phenylamine (53.5% of theory) are obtained in the form of a brownish liquid (trans/cis ratio: 3:1).

### EXAMPLE P3

### Preparation of 2-(1′-methyl-bicyclopropyl-2-yl)-phenylamine

A mixture of 10 g of 2′-(2-bromophenyl)-1-methyl-bicyclopropyl (42 mmol, trans/cis mixture, with trans/cis ratio: 2:1), 66 g of ammonia gas (3.9 mol), 600 mg of Cu₂O (4.2 mmol) and 65 ml of ethylene glycol is heated at a temperature of 150° C. for 36 hours in an autoclave at a pressure of 75-85 bar. After evaporation of the ammonia, 200 ml of ethyl acetate are added. The organic phase is washed with water and dried over sodium sulfate and concentrated by evaporation. For separation of secondary products, chromatography is carried out on silica gel (eluant: ethyl acetate/hexane 1:4). After removal of the eluant, 2-(1′-methyl-bicyclopropyl-2-yl)-phenylamine is obtained in a yield of 80% of theory in the form of a brownish liquid (trans/cis ratio: 2:1).

The following compounds of formula I can be prepared on the basis of the above Examples:

TABLE 1
Compounds of formula I

| | |
|---|---|
| | (I) |
| 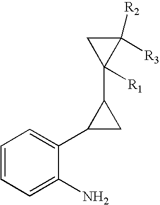 | |

| Comp. No. | R₁ | R₂ | R₃ |
|---|---|---|---|
| A1 | H | H | H |
| A2 | CH₃ | H | H |
| A3 | H | CH₃ | H |
| A4 | H | H | CH₃ |
| A5 | CH₃ | CH₃ | H |
| A6 | CH₃ | H | CH₃ |
| A7 | H | CH₃ | CH₃ |
| A8 | CH₃ | CH₃ | CH₃ |

The following compounds of formula II are suitable for use in the process according to the invention:

TABLE 2
Compounds of formula II

| | |
|---|---|
| | (II) |
| 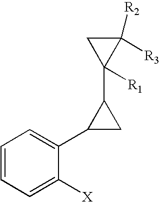 | |

| Comp. No. | X | R₁ | R₂ | R₃ |
|---|---|---|---|---|
| B1 | Br | H | H | H |
| B2 | Br | CH₃ | H | H |
| B3 | Br | H | CH₃ | H |
| B4 | Br | H | H | CH₃ |
| B5 | Br | CH₃ | CH₃ | H |
| B6 | Br | CH₃ | H | CH₃ |
| B7 | Br | H | CH₃ | CH₃ |
| B8 | Br | CH₃ | CH₃ | CH₃ |
| B9 | Cl | H | H | H |
| B10 | Cl | CH₃ | H | H |
| B11 | Cl | H | CH₃ | H |
| B12 | Cl | H | H | CH₃ |
| B13 | Cl | CH₃ | CH₃ | H |
| B14 | Cl | CH₃ | H | CH₃ |
| B15 | Cl | H | CH₃ | CH₃ |
| B16 | Cl | CH₃ | CH₃ | CH₃ |

The present invention makes it possible for ortho-alkyl-substituted halobenzenes to be aminated in high yields and at low cost.

The starting materials for the process of the present invention are distinguished by ready accessibility and ease of handling and are also inexpensive.

The present invention makes it possible to use copper-containing compounds in catalytic amounts, preferably in a ratio of from 1:5 to 1:100 relative to compounds of formula II, especially in a ratio of from 1:10 to 1:20 relative to compounds of formula II. As a result, only a small amount of copper-containing catalyst is required, which renders the process especially inexpensive.

In a preferred embodiment of the invention, the reaction period for the reaction according to the invention is from 6 to 24 hours, especially from 6 to 18 hours. By virtue of those short reaction periods, this embodiment constitutes a particularly economically interesting variant of the process according to the invention.

As regards the selection of suitable reaction conditions, compounds of formula IIa (trans) react more quickly to form compounds of formula Ia (trans) than do compounds of formula IIb (cis) to form compounds of formula Ib (cis). For example, under the reaction conditions of Preparation Example 1 (0.1 equivalent of Cu₂O, 100 equivalents of ammonia, ethylene glycol as solvent and a reaction temperature of 150° C.), compounds of formula IIa (trans) wherein X is bromine and R₁, R₂ and R₃ are hydrogen were found to have reaction rates 1.7 times faster than compounds of formula IIb (cis) wherein X is bromine and R₁, R2 and R₃ are hydrogen. For this reason, in the preparation of compounds of formula I having an increased content of compounds of formula Ia (trans) or in the preparation of high-purity compounds of formula Ia (trans) especially short reaction times can be achieved. By virtue of those especially short reaction times, such an embodiment constitutes a particularly economically interesting variant of the process according to the invention for the preparation of compounds of formula I having an increased content of compounds of formula Ia (trans) or for the preparation of high-purity compounds of formula Ia (trans).

When the process according to the invention is used with ethylene glycol as solvent, in addition to the formation of the desired compounds of formula I, small amounts of secondary products in which substitution with ethylene glycol instead of ammonia has taken place can also be formed. Because compounds of formula I are valuable intermediates in the preparation of amide fungicides, as described, for example, in WO 03/074491, small amounts of impurities based on such secondary products can accordingly also occur in the amide fungicides themselves. For example, in the preparation of the amide fungicide of formula C1

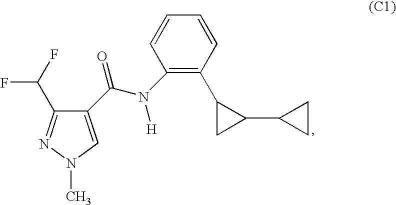

using a compound of formula A1

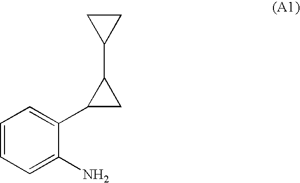

that has been prepared in accordance with the process of the invention using ethylene glycol as solvent, and using the preparation procedure described in WO 03/074491, such as, for example, the reaction of the aniline of formula Al with an acid chloride of formula C2

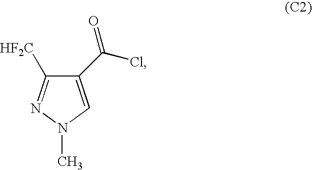

small amounts of the impurity C3

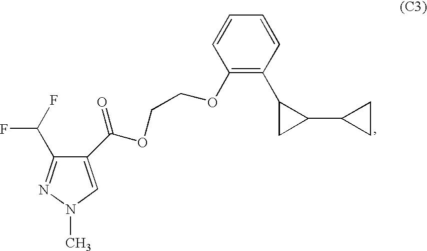

may be formed alongside the desired amide fungicide of formula C1.

What is claimed is:

1. A process for the preparation of a compound of formula I 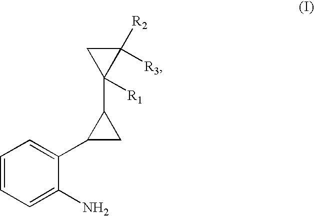
wherein R₁, R₂ and R₃ are each independently of the others hydrogen or methyl, wherein a compound of formula II 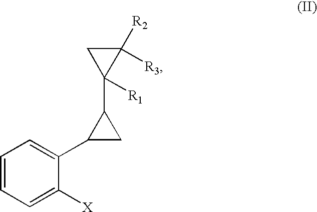
wherein R₁, R₂ and R₃ are as defined for formula I and X is bromine or chorine, is reacted with ammonia in the presence of a catalytic amount of at least one copper-containing compound.

2. A process according to claim 1, wherein a copper(I) compound or a mixture of copper(I) compounds is used as copper-containing compound.

3. Use of ammonia in the presence of a catalytic amount of at least one copper-containing compound in the amination of a compound of formula II 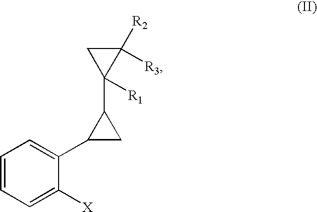
wherein R₁, R₂ and R₃ are as defined in claim 1 and X is bromine or chlorine.

4. A process for the amination of a compound of formula II 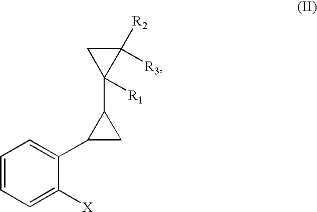
wherein R₁, R₂ and R₃ are as defined in claim 1 and X is bromine or chlorine, by using ammonia as aminating agent and a catalytic amount of at least one copper-containing compound.
